(19) United States
(12) Patent Application Publication
(10) Pub. No.: US 2010/0087459 A1
(43) Pub. Date: Apr. 8, 2010

(54) FORMS OF LAPATINIB COMPOUNDS AND PROCESSES FOR THE PREPARATION THEREOF

(76) Inventors: Leonid METSGER, Beer-Sheva (IL); Ariel MITTELMAN, St., Elad (IL); Slavik YURKOVSKI, Kiryat-Gat (IL)

Correspondence Address: KENYON & KENYON LLP, ONE BROADWAY, NEW YORK, NY, 10004, US

(21) Appl. No.: 12/548,103

(22) Filed: Aug. 26, 2009

Related U.S. Application Data
(60) Provisional application No. 61/091,981, filed on Aug. 26, 2008.
(60) Provisional application No. 61/093,614, filed on Sep. 2, 2008.
(60) Provisional application No. 61/103,404, filed on Oct. 7, 2008.
(60) Provisional application No. 61/185,000, filed on Jun. 8, 2009.
(60) Provisional application No. 61/185,439, filed on Jun. 9, 2009.

(51) Int. Cl. A61K 31/517; C07D 239/72; A61P 35/00

(52) U.S. Cl. 514/266.24; 544/293

(57) ABSTRACT

The present invention provides novel crystalline and amorphous lapatinib compounds and processes for preparing them

### RELATED APPLICATIONS

This application claims the benefit under 35 U.S.C. §119(e) of U.S. Provisional Application Nos. 61/091,981, filed Aug. 26, 2008; 61/093,614, filed Sep. 2, 2008; 61/103,404, filed Oct. 7, 2008; 61/185,000, filed Jun. 8, 2009; 61/185,439, filed Jun. 9, 2009; each of which is incorporated herein by reference in its entirety.

### FIELD OF THE INVENTION

The invention encompasses new crystalline and amorphous forms of lapatinib compounds, and processes for the preparation thereof.

### BACKGROUND OF THE INVENTION

Lapatinib ditosylate, N-[3-chloro-4-[(3-fluorophenyl)methoxy]phenyl]-6-[5-[(2-methylsulfonylethylamino)methyl]-2-furyl]quinazolin-4-amine ditosylate, has the following chemical structure:

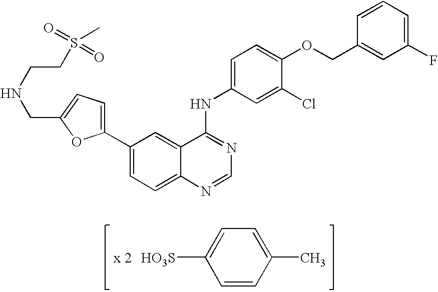

Lapatinib ditosylate is currently marketed in the United States under the tradename TYKERB® by GlaxoSmithKline. It was approved by the FDA as a drug for use in patients with advanced metastatic breast cancer.

Lapatinib ditosylate is described in PCT publications WO1999/035146, WO2002/002552, WO2005/046678, WO2006/113649, WO1998/002437, WO2001/004111, WO1996/009294, WO2002/056912, WO2005/105094, WO2005/120504, WO2005/120512, WO2006/026313, and WO2006/066267.

Two polymorphs of lapatinib ditosylate, anhydrous and monohydrate forms are described in U.S. Pat. No. 7,157,466 (WO 02/002552). Lapatinib hydrochloride salt is described in U.S. Pat. No. 6,727,256 (WO 99/35146).

The following lapatinib salts (including hydrates) are described in WO2008154469: Di and mono esylate, di and mono mesylate, di and mono L-lactate, di and mono L-malate, dimaleate, dibenzoate, di and mono L-tartrate, monocitrate, fumarate, besylate, hydrobromide, salicylate, succinate and edisylate. There is no physical characterization (XRD, thermal analysis, spectroscopy etc.) for the hydrobromide, L-tartrate, fumarate, edisylate, salicylate and Di-L-malate salts.

### SUMMARY OF THE INVENTION

The present invention encompasses novel solid crystalline and amorphous forms of lapatinib compounds; processes for preparing thereof, and pharmaceutical compositions containing one or more of these forms.

### BRIEF DESCRIPTION OF THE FIGURES

FIG. 1.1 shows a powder X-ray diffraction pattern for Form M1 of Lapatinib monotosylate.

FIG. 1.2 shows a solid-state ¹³C NMR spectrum of Form M1 of Lapatinib monotosylate.

FIG. 1.3 shows a solid-state ¹³C NMR spectrum of Form M1 of Lapatinib monotosylate.

FIG. 2 shows a powder X-ray diffraction pattern for Form F1 of Lapatinib fumarate.

FIG. 3 shows a powder X-ray diffraction pattern for Form S1 of Lapatinib succinate.

FIG. 4.1 shows a powder X-ray diffraction pattern for Form U1 of Lapatinib sulfate.

FIG. 4.2 shows a powder X-ray diffraction pattern for Form U2 of Lapatinib sulfate.

FIG. 4.3 shows a powder X-ray diffraction pattern for Form U3 of Lapatinib sulfate.

FIG. 4.4 shows a powder X-ray diffraction pattern for wet Form U4 of Lapatinib sulfate.

FIG. 4.5 shows a powder X-ray diffraction pattern for dry Form U4 of Lapatinib sulfate.

FIG. 4.6 shows a powder X-ray diffraction pattern for wet Form U5 of Lapatinib sulfate.

FIG. 4.7 shows a powder X-ray diffraction pattern for dry Form U5 of Lapatinib sulfate.

FIG. 4.8 shows a powder X-ray diffraction pattern for wet Form U6 of Lapatinib sulfate.

FIG. 4.9 shows a powder X-ray diffraction pattern for dry Form U6 of Lapatinib sulfate.

FIG. 4.10 shows a powder X-ray diffraction pattern for dry Form U6 of Lapatinib sulfate.

FIG. 4.11 shows a powder X-ray diffraction pattern for wet Form U7 of Lapatinib sulfate.

FIG. 4.12 shows a powder X-ray diffraction pattern for amorphous Lapatinib sulfate.

FIG. 4.13 shows a powder X-ray diffraction pattern for amorphous Lapatinib sulfate.

FIG. 5.1 shows a powder X-ray diffraction pattern for Form C1 of Lapatinib di-hydrochloride.

FIG. 5.2 shows a powder X-ray diffraction pattern for Form C1 of Lapatinib di-hydrochloride with higher crystallinity.

FIG. 5.3 shows a powder X-ray diffraction pattern for amorphous Lapatinib di-hydrochloride.

FIG. 6 shows a powder X-ray diffraction pattern for amorphous Lapatinib di-hydrobromide.

FIG. 7 shows a powder X-ray diffraction pattern for Form P2 of Lapatinib phosphate.

FIG. 8 shows a powder X-ray diffraction pattern for Form P3 of Lapatinib phosphate.

FIG. 9 shows a powder X-ray diffraction pattern for Form P4 of Lapatinib phosphate.

FIG. 10 shows a powder X-ray diffraction pattern for Form L1 of Lapatinib maleate.

FIG. 11 shows a powder X-ray diffraction pattern for amorphous Lapatinib tartrate.

### DETAILED DESCRIPTION OF THE INVENTION

The present invention relates to the solid state physical properties of Lapatinib compounds. These properties can be influenced by controlling the conditions under which Lapatinib compounds are obtained in solid forms. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. These conformational and orientation factors in turn result in particular intramolecular interactions such that different polymorphic forms may give rise to distinct spectroscopic properties that may be detectable by powder X-ray diffraction, solid state ¹³C NMR spectrometry and infrared spectrometry. A particular polymorphic form may also give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others.

The discovery of new solid states of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional processes for preparation of pharmaceutically acceptable compounds of Lapatinib either in a crystalline or in an amorphous form.

As used herein, the terms “Lapatinib monotosylate” “Lapatinib fumarate”, “Lapatinib succinate”, “Lapatinib sulfate”, “Lapatinib hydrochloride”, “lapatinib hydrobromide”, “Lapatinib phosphate”, “Lapatinib maleate”, and “Lapatinib tartrate” include any solid state composition of lapatinib base and p-toluenesulfonic acid, fumaric acid, succinic acid, sulfuric acid, hydrochloric acid, hydrobromic acid, phosphoric acid, maleic acid, and tartaric acid, respectively, for example: a salt, a co-crystal, or a solid mixture of base and acid.

As used herein, the term “Lapatinib base Form X” refers to a crystalline Form of Lapatinib base characterized by a PXRD pattern with peaks at about 6.9, 11.4, and 16.0±0.2 degrees 2-theta, and at least two peaks at positions selected from the group consisting of 4.6, 20.0, 21.4, 22.9, 25.2, 27.5, and 32.2±0.2 degrees 2-theta. Lapatinib base Form X can be obtained, for example, by forming a slurry of Lapatinib ditosylate and acetonitrile; and adding an inorganic base to obtain Lapatinib base Form X.

As used herein, room temperature (RT) refers to a temperature of about 20° C. to about 35° C.

As used herein, the term volume (“V”) refers to ml of solvent per gram of solute. For example, 30 V means 30 ml solvent per one gram of compound.

In the invention, in embodiments in which sulfuric acid is used, the sulfuric acid used is often concentrated sulfuric acid. The concentrations of sulfuric acid used is preferably 85-99%, more preferably 90-99%, most preferably 95-99% and particularly 98%. In the invention, in embodiments in which hydrochloric acid is used, the hydrochloric acid used is preferably an aqueous solution of about 30-38%, more preferably 30-35% and most preferably about 32% hydrochloric acid. In the invention, in embodiments in which hydrobromic acid is used, the hydrobromic acid used is preferably an aqueous solution of about 35-50%, more preferably about 45-50%, and most preferably about 46-49%, especially about 48% hydrobromidic acid. In the invention, in embodiments in which phosphoric acid is used, the phosphoric acid is preferably an aqueous solution of about 70-90%, preferably about 80 to about 88%, and most preferably about 83-86%, particularly about 85% phosphoric acid.

In one embodiment, the present invention encompasses Lapatinib monotosylate. Preferably, the Lapatinib monotosylate is solid, more preferably, it is crystalline.

In another embodiment, the invention encompasses crystalline Form M1 of Lapatinib monotosylate characterized by data selected from the group consisting of: a PXRD pattern with peaks at about 5.3, 6.0, 7.4, 10.0, and 17.7±0.2 degrees 2-theta; a PXRD pattern with peaks at about 5.3, 6.0, and 7.4±0.2 degrees 2-theta, and at least two peaks selected from the group consisting of 17.7, 18.6, 19.9, 23.3 and 23.9±0.2 degrees 2-theta; a PXRD pattern with peaks at about 5.3, 6.0, 7.4, 10.0, 17.7, 19.8, 21.5, 23.3, 23.9 and 26.7±0.2 degrees 2-theta; a solid-state ¹³C NMR spectrum with signals at about 113.3, 127.0 and 129.4±0.2 ppm; and a solid-state ¹³C NMR spectrum having chemical shifts in the range of 100 to 180 ppm of about 3.0, 16.7 and 19.1±0.1 ppm relative to a lowest chemical shift in the range of 110 to 180 ppm, wherein the lowest chemical shift is typically at about 110.3±0.1 ppm.

In particular, Form M1 is characterized by a PXRD pattern with peaks at about 5.3, 6.0, 7.4, 10.0, and 17.7±0.2 degrees 2-theta.

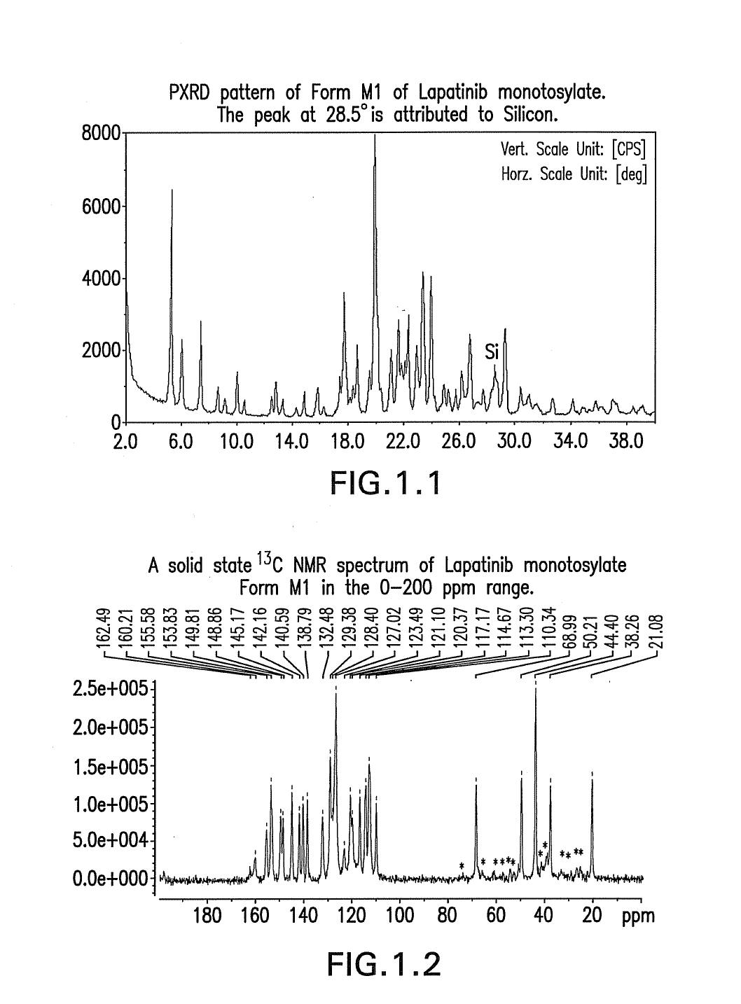

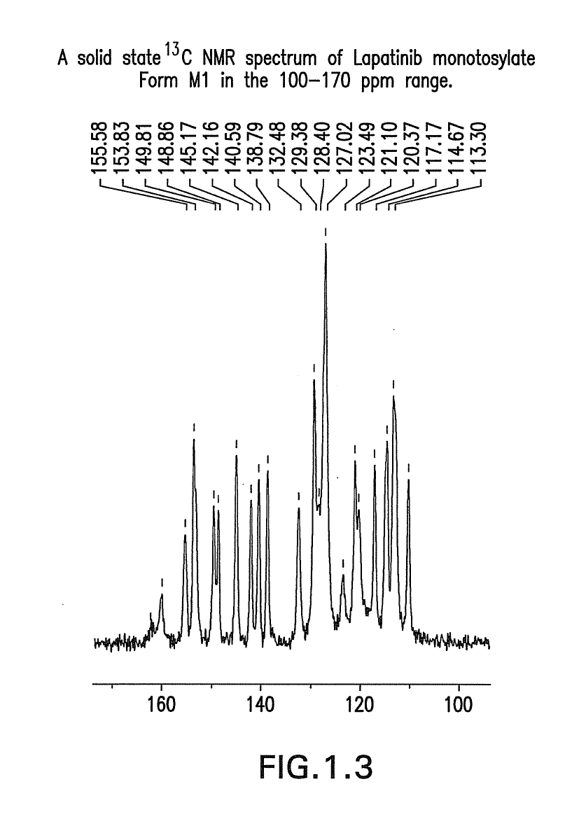

In another embodiment, the present invention encompasses crystalline Form M1 of Lapatinib monotosylate as characterized by a PXRD pattern illustrated in FIG. 1.1, and by solid-state ¹³C NMR spectrum illustrated in FIGS. 1.2 and 1.3.

This crystalline Form M1 is found to possess a high stability in commonly used solvents, which are used for production and especially for formulation of the pharmaceutical product, as well as a high-temperature polymorphic stability.

The present invention further relates to Lapatinib fumarate in a crystalline form.

The crystalline form, designated Form F1, of Lapatinib fumarate can be characterized by a PXRD pattern with peaks at about 17.5, 20.4, and 21.1±0.2 degrees 2-theta, and at least two peaks selected from the group consisting of 21.8, 22.5, 23.4, 25.3 and 26.8±0.2 degrees 2-theta.

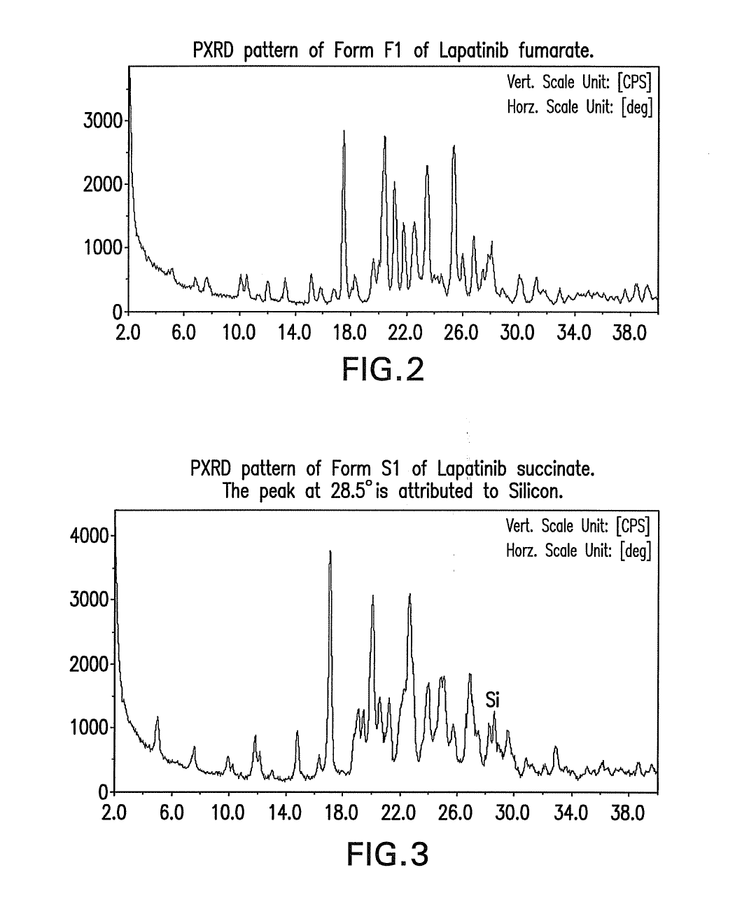

Crystalline Form F1 of Lapatinib fumarate can be further characterized by a PXRD pattern illustrated in FIG. 2.

In another embodiment, the present invention relates to Lapatinib sulfate. Preferably, the Lapatinib sulfate is solid, more preferably, it is crystalline.

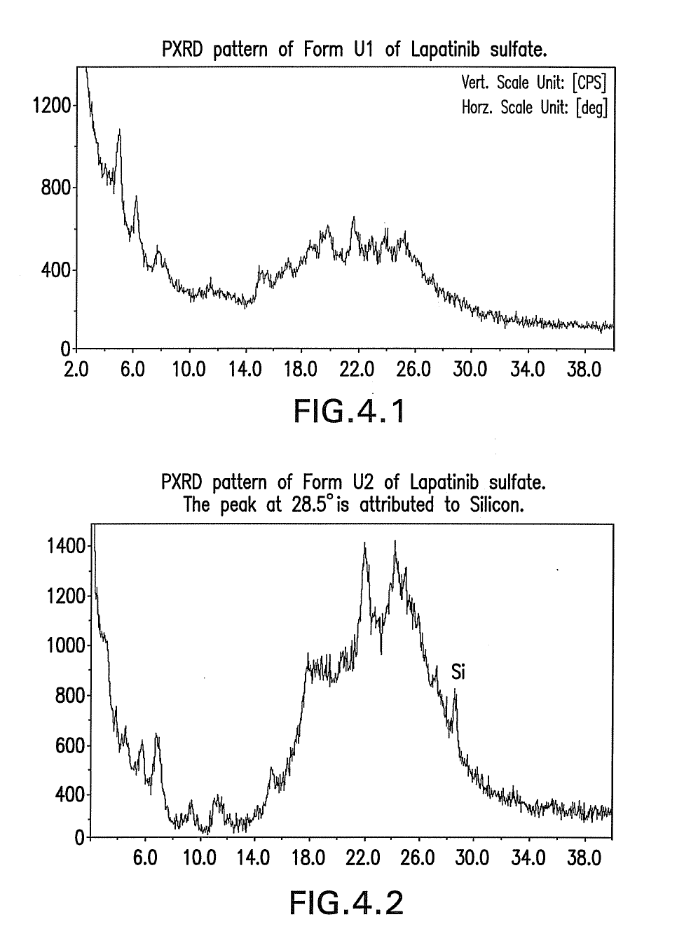

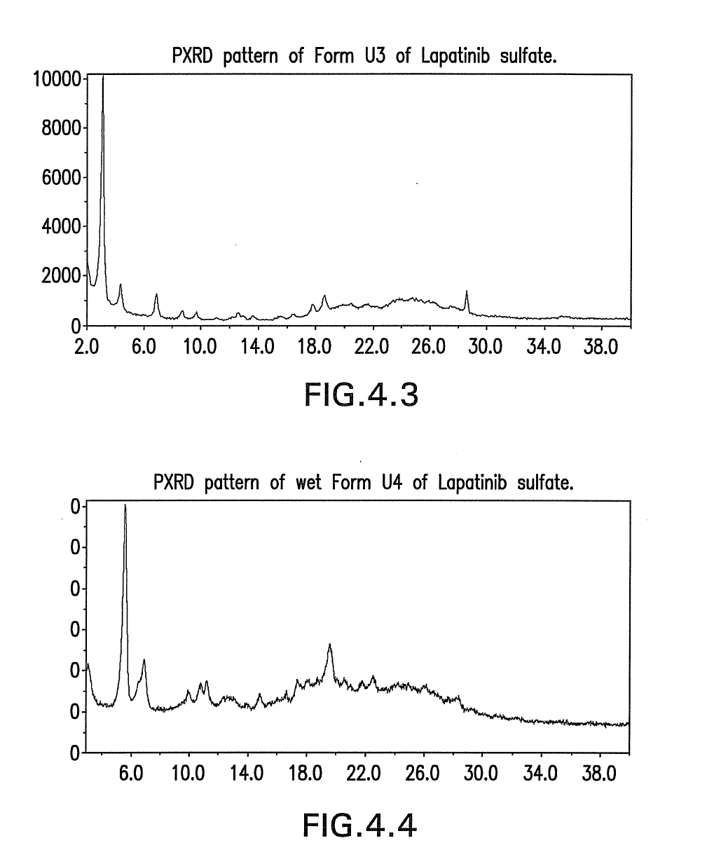

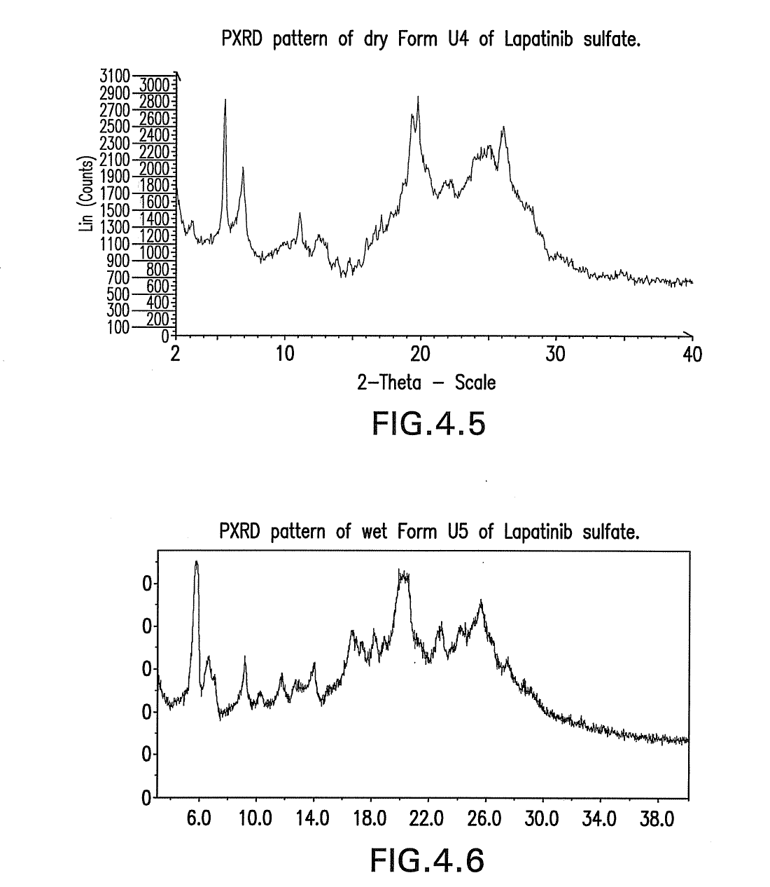

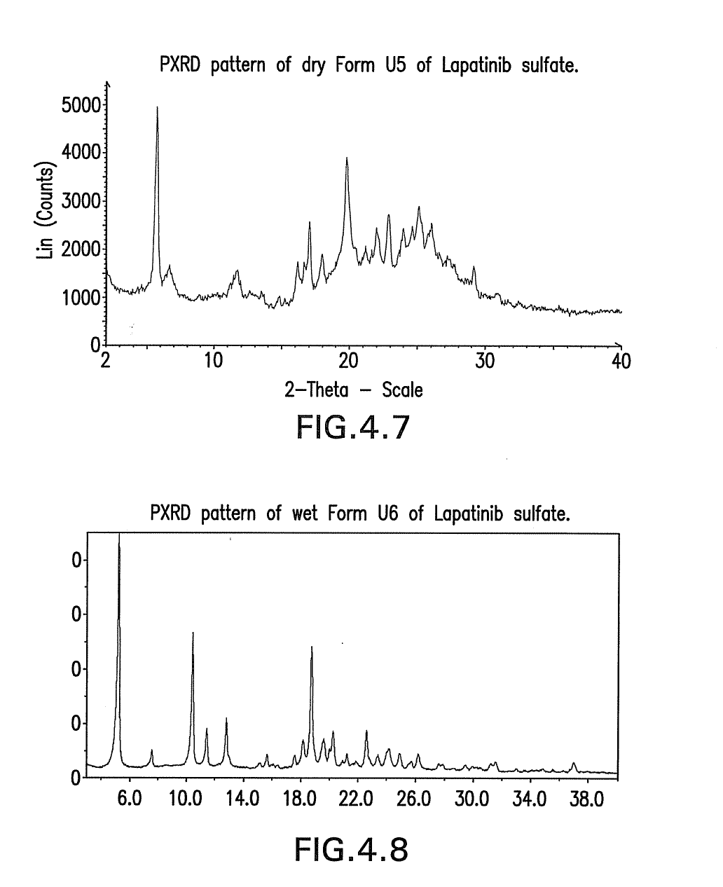

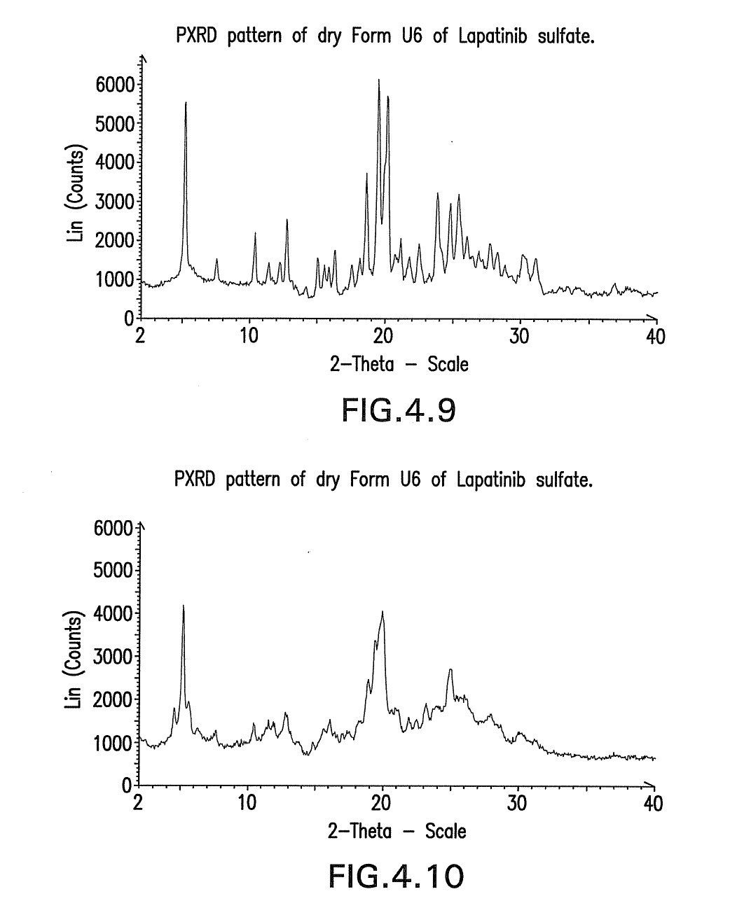

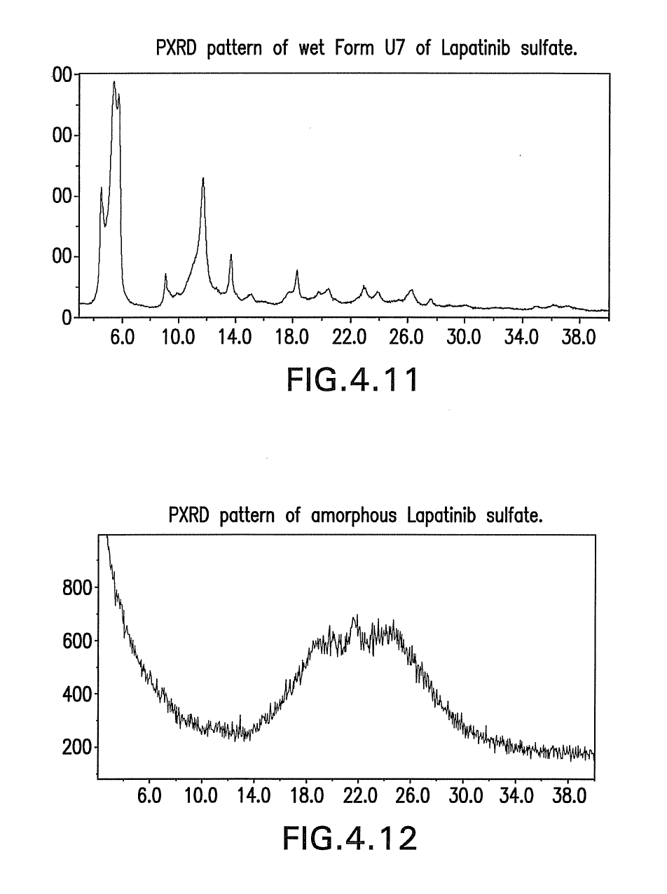

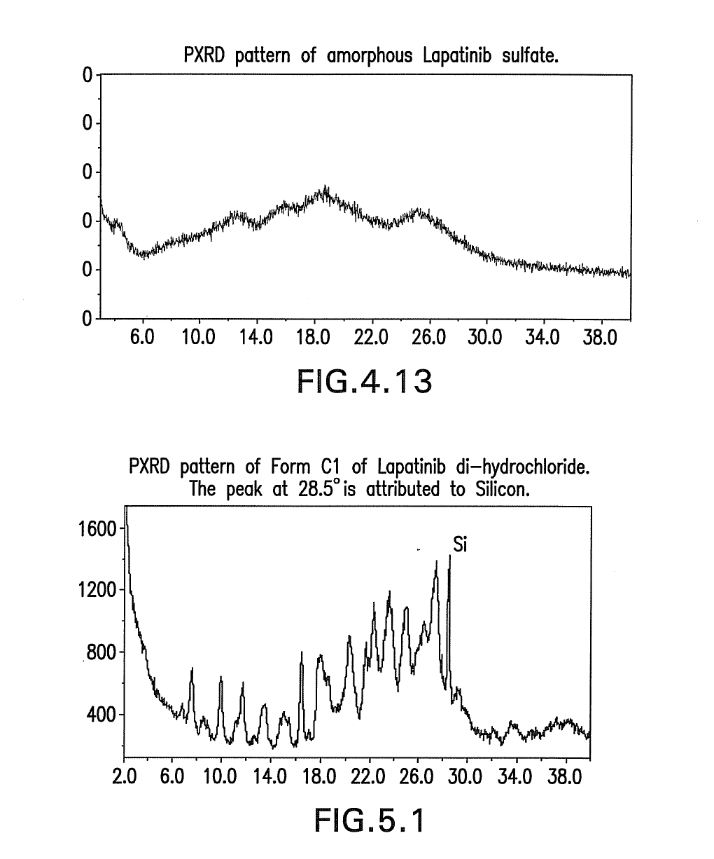

The invention further relates to crystalline Form U1 of Lapatinib sulfate, which can be characterized by a PXRD pattern with peaks at about 5.0, 6.2, and 21.6±0.2 degrees 2-theta, and at least two peaks selected from the group consisting of 7.9, 17.0, 19.8, 23.8, and 25.2±0.2 degrees 2-theta. Form U1 can be further characterized by a PXRD pattern illustrated in FIG. 4.1.

The invention further relates to crystalline Form U2 of lapatinib sulfate, which can be characterized by a PXRD pattern with peaks at about 6.9, 21.9, and 24.1±0.2 degrees 2-theta, and at least two peaks selected from the group consisting of 4.5, 5.7, 9.3, 15.2 and 27.2±0.2 degrees 2-theta. Form U2 of Lapatinib sulfate can be further characterized by a PXRD pattern illustrated in FIG. 4.2.

The invention further relates to crystalline Form U3 of Lapatinib sulfate, which can be characterized by a PXRD pattern with peaks at about 3.1, 6.8, 9.6, 12.5 and 18.5±0.2 degrees 2-theta. Form U3 can be further characterized by a PXRD pattern with peaks at about 3.1, 4.3, 6.8, 8.6, 9.6, 12.5, 13.6, 16.4, 17.7 and 18.6±0.2 degrees 2-theta. Form U3 of Lapatinib sulfate can be further characterized by a PXRD pattern illustrated in FIG. 4.3.

The invention further relates to crystalline Form U4 of Lapatinib sulfate, which can be characterized by a PXRD pattern with peaks at about 5.6, 6.9, 11.1, 14.8 and 19.7±0.3 degrees 2-theta. Form U4 of Lapatinib sulfate can be further characterized by a PXRD pattern illustrated in FIGS. 4.4 and 4.5.

The invention further relates to crystalline Form U5 of Lapatinib sulfate characterized by a PXRD pattern with peaks at about 5.7, 6.6, 11.7 and 17.9±0.2 degrees 2-theta. Form U5 can be further characterized by a PXRD pattern with peaks at about 5.7, 6.6, 11.7, 16.1, 17.0, 17.9, 19.7, 21.9 and 22.8±0.2 degrees 2-theta. Form U5 can be further characterized by a PXRD pattern with peaks at about 5.7, 6.6, 9.0, 11.7, 13.8, 16.4, 17.9, 22.5 and 25.4±0.2 degrees 2-theta. Form U5 of Lapatinib sulfate can be further characterized by a PXRD pattern illustrated in FIGS. 4.6, and 4.7.

In another embodiment, the invention encompasses crystalline Form U6 of Lapatinib sulfate characterized by a PXRD pattern with peaks at about 5.2, 10.4, 11.4 and 12.7±0.2 degrees 2-theta.

Lapatinib sulfate Form U6 can be further characterized by a PXRD pattern with peaks at about 5.2, 7.5, 10.4, 11.4, 12.7, 18.1, 18.6, 19.5, 20.1 and 22.5±0.2 degrees 2-theta.

In another embodiment, the present invention encompasses crystalline Form U6 of Lapatinib sulfate as characterized by a PXRD pattern illustrated in FIGS. 4.8 and 4.9.

The invention further relates to crystalline Form U5 of Lapatinib sulfate characterized by a PXRD pattern with peaks at about 4.6, 9.1, 11.7, 13.7, and 18.2±0.2 degrees 2-theta. Lapatinib sulfate Form U7 can be further characterized by a PXRD pattern with peaks at about 4.6, 5.5, 5.8, 9.1, 11.7, 13.7, 18.2, 20.4 and 22.9±0.2 degrees 2-theta. Lapatinib sulfate Form U7 can be further characterized by a PXRD pattern illustrated in FIG. 4.11.

In another embodiment, the present invention encompasses amorphous lapatinib sulfate as characterized by a PXRD pattern illustrated in FIGS. 4.12 and 4.13.

The present invention further relates to solid Lapatinib di-hydrochloride. Preferably, the Lapatinib di-hydrochloride is crystalline.

In another embodiment, the invention encompasses crystalline Form C1 of lapatinib di-hydrochloride characterized by data selected by the group consisting of: a PXRD pattern with peaks at about 7.7, 9.9, 11.8, 13.6 and 16.5±0.2 degrees 2-theta; a PXRD pattern with peaks at about 7.7, 9.9, 11.8, 13.6, 16.5, 17.9, 18.6, 20.5, 22.4 and 23.7±0.2 degrees 2-theta; a PXRD pattern with peaks at about 7.6, 10.0, and 11.8±0.2 degrees 2-theta, and at least two peaks selected from the group consisting of 16.5, 18.1, 20.4, 22.3 and 23.6±0.2 degrees 2-theta; a solid-state ¹³C NMR spectrum with signals at about 113.0, 131.0 and 152.3±0.2 ppm; and a solid-state ¹³C NMR spectrum having chemical shifts in the range of 100 to 180 ppm of about 3.5, 21.5 and 42.8±0.1 ppm relative to a lowest chemical shift in the range of 110 to 180 ppm, wherein the lowest chemical shift is typically at about 109.5±0.1 ppm.

In a particular embodiment, Form C1 is characterized by a PXRD pattern with peaks at about 7.7, 9.9, 11.8, 13.6 and 16.5±0.2 degrees 2-theta.

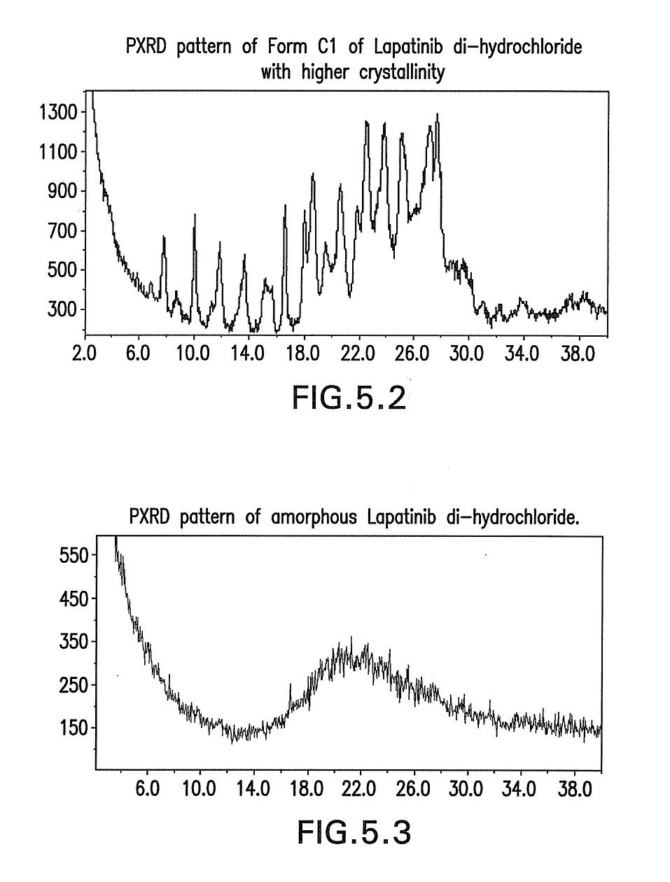

In another embodiment, the present invention encompasses crystalline Form C1 of Lapatinib di-hydrochloride as characterized by a PXRD pattern illustrated in FIGS. 5.1 and 5.2, and by a solid-state ¹³C NMR spectrum illustrated in FIGS. 5.4 and 5.5.

In another embodiment, the present invention encompasses amorphous Lapatinib di-hydrochloride as characterized by a PXRD pattern illustrated in FIG. 5.3.

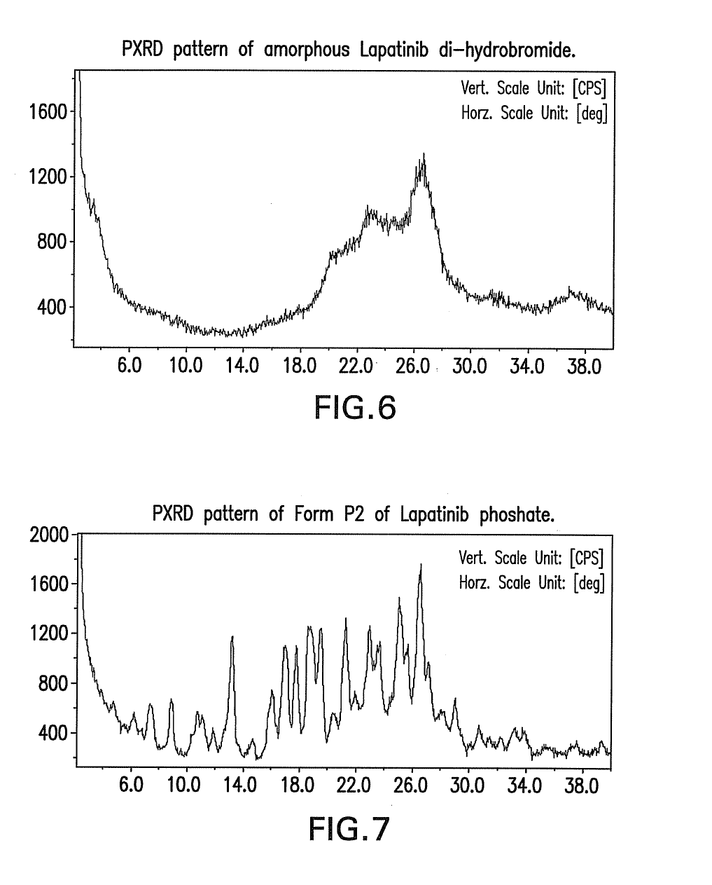

In another embodiment, the present invention encompasses amorphous Lapatinib di-hydrobromide as characterized by a PXRD pattern illustrated in FIG. 6.

In another embodiment, the present invention encompasses Lapatinib phosphate. Preferably, the Lapatinib phosphate is solid, more preferably, it is crystalline.

In another embodiment, the invention encompasses crystalline Form P2 of lapatinib phosphate characterized by a PXRD pattern with peaks at about 13.1, 16.8, and 17.6±0.2 degrees 2-theta, and at least two peaks selected from the group consisting of 18.6, 19.4, 21.1, 22.8, and 25.0±0.2 degrees 2-theta.

In another embodiment, the present invention encompasses crystalline Form P2 of Lapatinib phosphate as characterized by a PXRD pattern illustrated in FIG. 7.

In another embodiment, the invention encompasses crystalline Form P3 of lapatinib phosphate characterized by a PXRD pattern with peaks at about 5.5, 16.3, and 17.6±0.2 degrees 2-theta, and at least two peaks selected from the group consisting of 7.7, 11.0, 11.3, 15.7, and 26.3±0.2 degrees 2-theta.

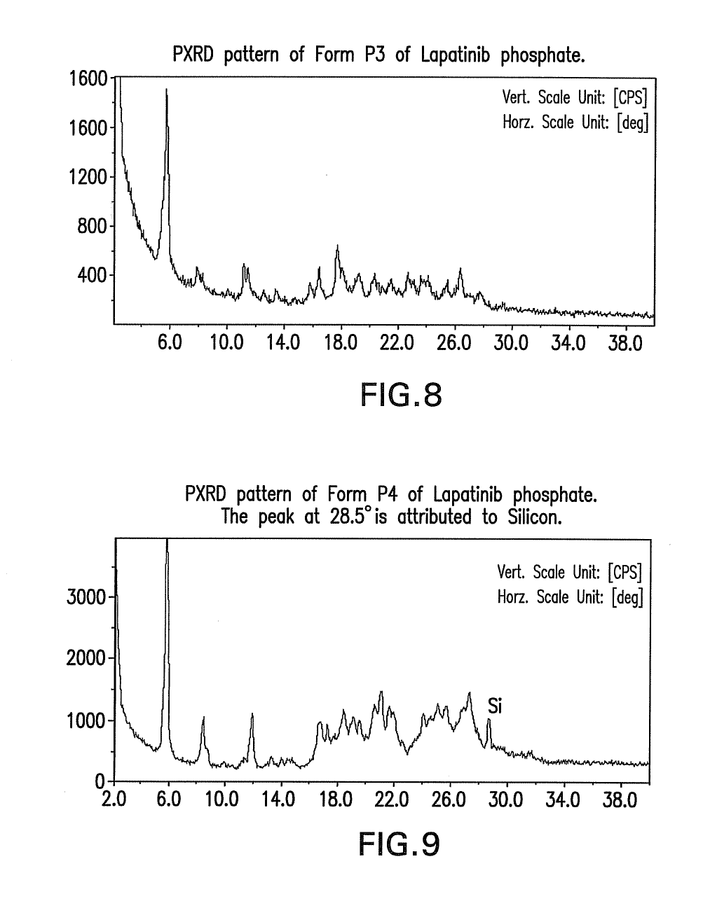

In another embodiment, the present invention encompasses crystalline Form P3 of Lapatinib phosphate as characterized by a PXRD pattern illustrated in FIG. 8.

In another embodiment, the invention encompasses crystalline Form P4 of lapatinib phosphate characterized by a PXRD pattern with peaks at about 5.5, 8.2, and 11.6±0.2 degrees 2-theta, and at least two peaks selected from the group consisting of 16.4, 17.0, 18.2, 20.4, 20.8±0.2 degrees 2-theta.

In another embodiment, the present invention encompasses crystalline Form P4 of Lapatinib phosphate as characterized by a PXRD pattern illustrated in FIG. 9.

The present invention further relates to amorphous lapatinib tartrate as characterized by a PXRD pattern illustrated in FIG. 11.

The present invention further relates to crystalline Form S1 of lapatinib succinate, which can be characterized by a PXRD pattern with peaks at about 17.0, 20.0, and 22.5±0.2 degrees 2-theta, and at least two peaks selected from the group consisting of 5.0, 7.6, 11.8, 14.8 and 24.8±0.2 degrees 2-theta. Form S1 is also characterized by a PXRD pattern illustrated in FIG. 3.

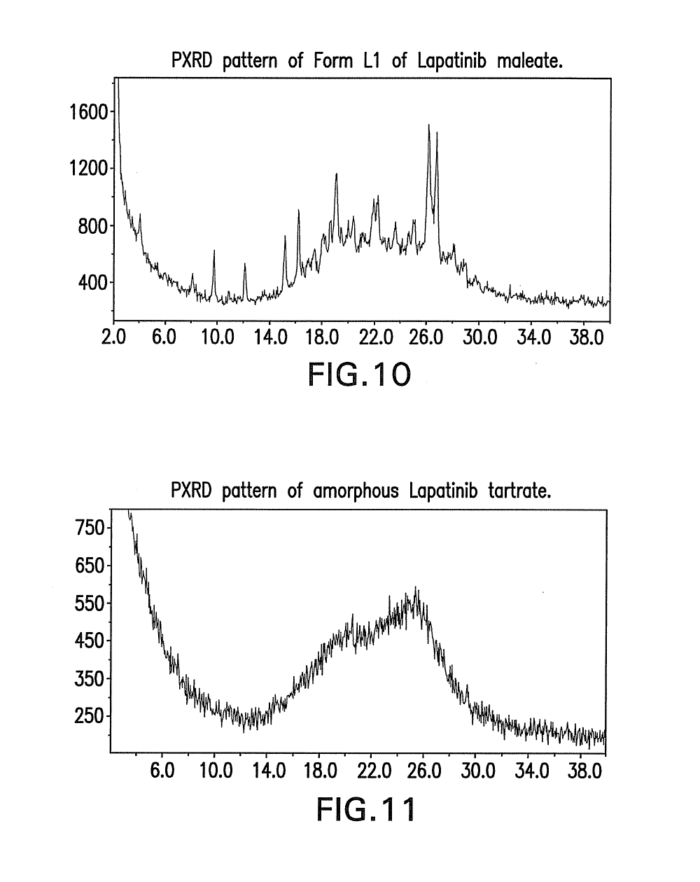

The present invention further relates to crystalline Form L1 of lapatinib maleate characterized by a PXRD pattern with peaks at about 9.7, 15.1, and 16.1±0.2 degrees 2-theta, and at least two peaks selected from the group consisting of 4.0, 12.0, 19.0, 26.1, and 26.7±0.2 degrees 2-theta. Form L1 is also characterized by a PXRD pattern illustrated in FIG. 10.

In one embodiment, the present invention encompasses a process for preparing crystalline Form M1 of Lapatinib monotosylate comprising forming a suspension of Lapatinib base with about 1 to about 1.2 equivalents of p-toluenesulfonic acid in methanol; and recovering the precipitate from the obtained slurry. Preferably the starting Lapatinib base in any embodiment of this process is Form X. Preferably in any embodiment of this process, about 1 equivalent of p-toluenesulfonic acid is used. In any embodiment of this process, about 20 to about 50 ml, preferably about 30 to about 60 ml and most preferably about 35 to about 45 ml, and especially about 40 ml of methanol is used per gram of Lapatinib base.

The suspension is preferably maintained at about 0° C. to about room temperature, e.g., at about 0° C. to about 35° C., more preferably about 10° C. to about room temperature, and most preferably, about room temperature; preferably, for about 16 hours to about 24 hours, more preferably for about 20 hours to about 24 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 40° C. Preferably, the obtained precipitate is dried for about 16 hours to about 72 hours, more preferably, for about 16 hours to about 48 hours, and most preferably, for about 16 hours to about 24 hours.

In another embodiment, the present invention encompasses a process for preparing amorphous Lapatinib di-hydrobromide comprising forming a suspension of lapatinib base with hydrobromic acid in methanol; and recovering the precipitate from the obtained slurry. Preferably in any embodiment of this process, the starting material is Lapatinib base Form X. Preferably in any embodiment of this process, the hydrobromic acid is an aqueous solution of about 35-50%, more preferably about 45-50%, and most preferably about 46-49%, especially about 48% hydrogen bromide. Preferably, in any embodiment of this process, the hydrobromic acid is used in an amount of about 1.95 to about 3, more preferably about 2 to about 2.5 equivalents and most preferably about 2 equivalents. Preferably, in any embodiment of this process, the methanol is used in an amount of about 10 to about 60, more preferably about 20 to about 40, most preferably about 25-35 and especially about 30 ml per gram of Lapatinib base.

The suspension is preferably maintained at about 0° C. to about room temperature, e.g., at about 0° C. to about 35° C., more preferably about 10° C. to about room temperature, and most preferably, about room temperature; preferably, for about 16 hours to about 24 hours, more preferably for about 20 hours to about 24 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 40° C. Preferably, the obtained precipitate is dried for about 16 hours to about 72 hours, more preferably, for about 16 hours to about 48 hours, and most preferably, for about 16 hours to about 24 hours.

In another embodiment, the present invention encompasses a process for preparing amorphous Lapatinib tartrate comprising forming a suspension of Lapatinib base with tartaric acid in methanol; and recovering the precipitate from the obtained slurry. Preferably in any embodiment of this process, the starting material is Lapatinib base Form X. Preferably, the tartaric acid is a racemic tartaric acid. Preferably in any embodiment of this process, the tartaric acid is used in an amount of about 0.99 to about 1.2, more preferably about 1 to about 1.1 equivalents and most preferably about 1 equivalent. Preferably, in any embodiment of this process, the methanol is used in an amount of about 10 to about 60, more preferably about 20 to about 40, most preferably about 25-35 and especially about 30 ml per gram of Lapatinib base.

The suspension is preferably maintained at about 0° C. to about room temperature, e.g., at about 0° C. to about 35° C., more preferably about 10° C. to about room temperature, and most preferably, at about room temperature; preferably, for about 16 hours to about 24 hours, more preferably for about 20 hours to about 24 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 40° C. Preferably, the obtained precipitate is dried for about 16 hours to about 72 hours, more preferably, for about 16 hours to about 48 hours, and most preferably, for about 16 hours to about 24 hours.

In another embodiment, the present invention encompasses a process for preparing crystalline Form L1 of Lapatinib maleate comprising forming a suspension of lapatinib base with maleic acid in methanol; and recovering the precipitate from the obtained slurry. Preferably in any embodiment of this process, the starting material is Lapatinib base Form X. Preferably in any embodiment of this process, the maleic acid is used in an amount of about 0.99 to about 1.2, more preferably about 1 to about 1.1 equivalents and most preferably about 1 equivalent. Preferably, in any embodiment of this process, the methanol is used in an amount of about 10 to about 60, more preferably about 20 to about 40, most preferably about 25-35 and especially about 30 ml per gram of Lapatinib base.

The suspension is preferably maintained at about room temperature to about reflux; preferably, for about 16 hours to about 24 hours, more preferably for about 20 hours to about 24 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 40° C. Preferably, the obtained precipitate is dried for about 16 hours to about 72 hours, more preferably, for about 16 hours to about 48 hours, and most preferably, for about 16 hours to about 24 hours.

In another embodiment, the present invention encompasses a process for preparing crystalline Form S1 of Lapatinib succinate comprising forming a suspension of Lapatinib base with succinic acid in methanol; and recovering the precipitate from the obtained slurry. Preferably in any embodiment of this process, the starting material is Lapatinib base Form X. Preferably in any embodiment of this process, the succinic acid is used in an amount of about 0.99 to about 1.2, more preferably about 1 to about 1.1 equivalents and most preferably about 1 equivalent. Preferably, in any embodiment of this process, the methanol is used in an amount of about 10 to about 60, more preferably about 20 to about 40, most preferably about 25-35 and especially about 30 ml per gram of Lapatinib base.

The suspension is preferably maintained at about 0° C. to about room temperature, more preferably about 10° C. to about room temperature, and most preferably, at about room temperature; preferably, for about 16 hours to about 24 hours, more preferably for about 20 hours to about 24 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 40° C. Preferably, the obtained precipitate is dried for about 16 hours to about 72 hours, more preferably, for about 16 hours to about 48 hours, and most preferably, for about 16 hours to about 24 hours.

The present invention also provides a process for preparing Lapatinib sulfate. Lapatinib sulfate may be prepared by combining Lapatinib and sulfuric acid to create a reaction mixture. Lapatinib sulfate forms in such reaction mixture through contact of Lapatinib with sulfuric acid. Preferably in any embodiment of this process, the starting material is Lapatinib base Form X. Preferably in any embodiment of this process, the sulfuric acid is concentrated (i.e. preferably 85-99%, more preferably 90-99%, most preferably 95-99% and particularly 98%) sulfuric acid. Preferably in any embodiment of this process, the sulfuric acid is used in an amount of about 0.99 to about 1.2, more preferably about 1 to about 1.1 equivalents and most preferably about 1 equivalent. Preferably, in any embodiment of this process, the solvent is used in an amount of about 10 to about 60, more preferably about 20 to about 40, most preferably about 25-35 and especially about 30 ml per gram of Lapatinib base.

In one embodiment, Lapatinib base, a solvent and sulfuric acid are combined to form a reaction mixture, followed by recovery of the Lapatinib sulfate from the mixture, fir example by filtration. The organic solvent present in the reaction mixture is preferably selected from the group consisting of C₁₋₈ alcohols. Preferably, the solvent is methanol. Preferably in any embodiment of this process, the starting material is lapatinib base Form X. Preferably in any embodiment of this process, the sulfuric acid is concentrated (i.e. preferably 85-99%, more preferably 90-99%, most preferably 95-99% and particularly 98%) sulfuric acid. Preferably in any embodiment of this process, the sulfuric acid is used in an amount of about 0.99 to about 1.2, more preferably about 1 to about 1.1 equivalents and most preferably about 1 equivalent. Preferably, in any embodiment of this process, the solvent is used in an amount of about 10 to about 60, more preferably about 20 to about 40, most preferably about 25-35 and especially about 30 ml per gram of lapatinib base.

In one embodiment, Lapatinib base, sulfuric acid and at least one solvent are combined to form a reaction mixture at about room temperature. The amount of sulfuric acid present in such reaction mixture is preferably to the point of saturation. Lapatinib sulfate then precipitates out of such mixture. Such precipitation may occur on its own or be induced. The reaction mixture may be stirred before, during or after precipitation.

The resulting precipitate from any of the above embodiments may be recovered by conventional techniques, such as filtration. The precipitate may be dried under ambient or reduced pressure, or elevated temperature. In one embodiment, the precipitate is dried at room temperature at a pressure of less than about 100 mmHg. Preferably, the obtained precipitate is dried for about 16 hours to about 72 hours, more preferably, for about 16 hours to about 48 hours, and most preferably, for about 16 hours to about 24 hours.

The Lapatinib sulfate of the invention can form different polymorphic forms.

The polymorphic forms of Lapatinib sulfate can be obtained directly from the reaction mixture or by further combining the Lapatinib sulfate with an organic solvent selected from C₁₋₈ alcohols, C₁₋₆ ketones, preferably, acetone, DMF (dimethylformamide), and DMA (dimethylacetamide).

In one specific embodiment, the present invention encompasses a process for preparing crystalline Form U6 of Lapatinib sulfate comprising forming a suspension of lapatinib sulfate in a solvent selected from the group consisting of DMF, and DMA; and recovering the precipitate from the obtained slurry. When DMA is used, the obtained precipitate is further dried. Preferably in any embodiment of this process, the solvent is used in an amount of about 10 to about 60, more preferably about 10 to about 30, most preferably about 15-25 and especially about 20 ml per gram of Lapatinib sulfate.

Preferably, the starting Lapatinib sulfate is Lapatinib sulfate Form U3.

The suspension is preferably maintained at about 0° C. to about room temperature, more preferably about 10° C. to about room temperature, and most preferably, at about room temperature; preferably, for about 3 hours to about 24 hours, more preferably for about 3 hours to about 12 hours, and most preferably, about 3 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 50° C. Preferably, the obtained precipitate is dried for about 16 hours to about 72 hours, more preferably, for about 16 hours to about 48 hours, and most preferably, for about 16 hours to about 24 hours.

The present invention also encompasses a process for preparing amorphous lapatinib sulfate comprising heating Lapatinib sulfate to melt, followed by cooling, preferably, to a temperature of about 0° C. to about room temperature. Preferably, the Lapatinib sulfate is heated to a temperature of about 150° C. Preferably, heating is carried out in air, under vacuum, or at the presence of an inert gas.

In another embodiment, the present invention encompasses another process for preparing amorphous Lapatinib sulfate comprising combining Lapatinib sulfate Form U3 with water to form a suspension; and maintaining the suspension for about 3 hours to about 24 hours, most preferably for about 3 hours, to obtain amorphous Lapatinib sulfate. Preferably in any embodiment of this process, the water is used in an amount of about 10 to about 60, more preferably about 10 to about 30, most preferably about 15-25 and especially about 20 ml per gram of Lapatinib sulfate.

The suspension is typically maintained at about 0° C. to about 35° C., more preferably at about 10° C. to about 35° C., and most preferably at about room temperature.

The present invention also encompasses a process for preparing Form C1 of lapatinib di-hydrochloride comprising providing a slurry of lapatinib di-hydrochloride in methanol; and recovering the precipitate. Preferably in any embodiment of this process, the methanol is used in an amount of about 10 to about 60, more preferably about 10 to about 30, most preferably about 15-25 and especially about 20 ml per gram of Lapatinib di-hydrochloride.

The Lapatinib di-hydrochloride starting material may optionally be formed in situ, by combining Lapatinib base and hydrochloric acid. Preferably in any embodiment of this process, the starting material is Lapatinib base Form X. Preferably the hydrochloric acid is an aqueous solution of about 30-38%, more preferably 30-35% and most preferably about 32% hydrogen chloride. Preferably, in any embodiment of this process, the hydrochloric acid is used in an amount of about 1.95 to about 3, more preferably about 2 to about 2.5 equivalents and most preferably about 2 equivalents.

The slurry is preferably maintained at about 0° C. to about room temperature, more preferably about 10° C. to about room temperature, and most preferably, at about room temperature; preferably, for about 3 hours to about 24 hours, more preferably for about 3 hours to about 12 hours, even more preferably, about 3 hours to about 5 hours, and most preferably, about 3 hours. Preferably, the suspension is then filtered.

The obtained precipitate can be further dried. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 50° C. Preferably, the obtained precipitate is dried for about 16 hours to about 72 hours, more preferably, for about 16 hours to about 48 hours, and most preferably, for about 16 hours to about 24 hours.

The present invention also encompasses a process for preparing amorphous Lapatinib di-hydrochloride comprising providing a slurry of Lapatinib di-hydrochloride preferably, Form C1, in dimethylsulfoxide (DMSO); and recovering the precipitate. Preferably, in any embodiment of this process, DMSO is used in an amount of about 10 to about 60, more preferably about 20 to about 40, most preferably about 25-35 and especially about 30 ml per gram of Lapatinib dihydrochloride.

The Lapatinib di-hydrochloride starting material may optionally be formed in situ, by combining Lapatinib base and hydrochloric acid. Preferably, in any embodiment of this process, the hydrochloric acid is used in an amount of about 1.95 to about 3, more preferably about 2 to about 2.5 equivalents and most preferably about 2 equivalents.

The suspension is preferably maintained at about 0° C. to about room temperature, more preferably about 10° C. to about room temperature, and most preferably, at about room temperature; preferably, for about 3 hours to about 24 hours, more preferably for about 3 hours to about 12 hours, even more preferably, about 3 hours to about 5 hours, and most preferably, about 3 hours. Preferably, the suspension is then filtered.

The obtained precipitate can be further dried to obtain crystalline Form C1 of Lapatinib di-hydrochloride. Drying can be carried out under a pressure of less than one atmosphere (reduced pressure), including a pressure of less than about 100 mmHg. Drying can be carried out by heating, with or without reducing the pressure, at about 40° C. to about 80° C., more preferably at about 40° C. to about 60° C., even more preferably at about 40° C. to about 50° C., and most preferably at about 50° C. Preferably, the obtained precipitate is dried for about 16 hours to about 72 hours, more preferably, for about 16 hours to about 48 hours, and most preferably, for about 16 hours to about 24 hours.

The present invention also encompasses a process for preparing crystalline Form P2 of lapatinib phosphate comprising providing a slurry of lapatinib base, one equivalent of phosphoric acid, and methanol; crystallizing lapatinib phosphate from the slurry to obtain lapatinib phosphate Form P2; and drying the precipitate at elevated temperature under reduced pressure. Preferably in any embodiment of this process, the Lapatinib base is Form X. Preferably, in any embodiment of this process, the phosphoric acid is used in an amount of about 0.99 to about 3, more preferably about 1 to about 2.5 equivalents and most preferably about 1 to about 2 equivalents. Preferably in any embodiment of this process, the phosphoric acid is preferably refers an aqueous solution of about 70-90%, preferably about 80 to about 88%, and most preferably about 83-86%, particularly about 85% phosphoric acid. Preferably, in any embodiment of this process, the methanol is used in an amount of about 10 to about 100, more preferably about 30 to about 80, most preferably about 40-60 and especially about 50 ml per gram of Lapatinib base.

The suspension is typically maintained at room temperature for about 9 hours. Preferably, the suspension is then filtered.

Drying can be carried out at a temperature of about 40° C. for about 16 hours to about 24 hours.

The present invention also encompasses a process for preparing crystalline Form P3 of lapatinib phosphate comprising providing a slurry of lapatinib base, two equivalents of phosphoric acid, and methanol; and crystallizing lapatinib phosphate from the slurry to obtain lapatinib phosphate Form P3.

The suspension is typically maintained at room temperature for about 20 hours to about 24 hours. Preferably, the suspension is then filtered.

The present invention also encompasses a process for preparing crystalline Form P4 of lapatinib phosphate comprising drying the crystalline Form P3 of Lapatinib phosphate at a temperature of about 40° C. under reduced pressure.

The crystalline form can be dried for about 16 hours to about 24 hours.

The present invention provides a pharmaceutical formulation comprising one or more of the described Lapatinib compounds. This pharmaceutical composition may additionally comprise at least one pharmaceutically acceptable excipient.

The invention further provides pharmaceutical formulations comprising one or more of the above described Lapatinib compounds, the lapatinib compounds made by the processes of the present invention, and one or more pharmaceutically acceptable excipients. The compositions of the invention include powders, granulates, aggregates and other solid compositions comprising the present invention forms of Lapatinib compounds.

The present invention also provides methods of treating metastatic breast cancer in a patient, preferably a human, by administrating to the patient a pharmaceutical composition comprising lapatinib compounds forms as described herein. Preferably, the pharmaceutical composition comprises a therapeutically effective amount of Lapatinib compound.

The present invention also provides the use of one or more of the described Lapatinib compounds for the manufacture of a pharmaceutical composition for the treatment of metastatic breast cancer.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### X-Ray Power Diffraction

X-Ray powder diffraction data was obtained by using methods known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2-40 degrees two-theta; scan mode: continuous scan; step size: 0.05°; and a rate of 3 deg/min. All peak positions are within ±0.2 degrees two-theta.

FIGS. 4.9 and 4.10 were obtained by using methods known in the art using a Bruker X-Ray powder diffractometer model D8 advance equipped with LynxEye.

Scan range: 2-40°. Step size: 0.05°. Time per step: 5.2 seconds. Cu Ka radiation.

FIGS. 4.4, 4.6, 4.8 and 4.11 were obtained by using methods known in the art using a Philips X'Pert PRO X-ray powder diffractometer, equipped with X'Celerator (2.022° 2Θ) detector. Scanning parameters: angle range: 3-40°, continuous scan, step size: 0.05 deg.; and a rate of 3 deg/min. Cu Ka radiation.

### Solid State NMR:

¹³C NMR at 125 MHz using Bruker Avance II+ 500
SB probe using 4 mm rotors
Magic angle was set using KBr
Homogeneity of magnetic field checked using adamantane
Parameters for Cross polarization optimized using glycine
Spectral reference set according to glycine as external standard (176.03 ppm for low field carboxyl signal).

### Preparation of Lapatinib Base Form X

In a 500 ml round-bottomed flask equipped with a mechanical stirrer and a condenser were added 10.8 g of wet Lapatinib ditosylate (i.e., Lapatinib ditosylate which was not dried in a vacuum oven) and 60 ml of acetonitrile. The resulted suspension was stirred at 40° C. for 1 hour. A solution of 1.24 g of sodium carbonate in 70 ml of water was added drop-wise during 5 minutes. The resulted yellow suspension was stirred at 40° C. for 1 hour. Then, it was stirred at 25° C. for 2 hours, at 5° C. for 0.5 hours. The product was filtered in vacuum and dried for 48 hours in a vacuum oven at 35° C. Yield: 6.2 g (91%)

### Example 1

To the mixture of 2 g solid Lapatinib-base form X and 0.66 g p-toluenesulfonic acid, 80 ml (40V) methanol was added to obtain a yellow suspension. The resulting suspension was stirred for 20 hours at 25° C. The cake thus obtained was dried for 16 hours at 40° C. in a vacuum oven, and identified as Form M1 of Lapatinib monotosylate.

### Example 2

To the mixture of 2 g solid Lapatinib-base form X and 0.4 g Fumaric acid, 60 ml (30V) methanol was added to obtain a yellow suspension. The resulting suspension was stirred for 20 hours at 25° C. The cake thus obtained was identified as Form F1 of Lapatinib fumarate.

### Example 3

To the mixture of 1.5 g solid Lapatinib-base form X and 0.3 g Succinic acid, 45 ml (30V) methanol was added to obtain a yellow suspension. The resulting suspension was stirred for 20 hours at 25° C. The cake thus obtained was dried for 16 hours at 40° C. in a vacuum oven, and identified as Form S1 of Lapatinib succinate.

### Example 4

To the mixture of 1.5 g solid Lapatinib-base form X and 0.136 ml H₂SO₄ 98%, 45 ml (30V) methanol was added to obtain a yellow suspension. The resulting suspension was stirred for 20 hours at 25° C. The cake thus obtained was identified as Form U1 of Lapatinib sulfate.

### Example 5

The cake obtained by Example 4 was dried for 16 hours at 40° C. in a vacuum oven, and identified as Form U2 of Lapatinib sulfate.

### Example 6

To the mixture of 1.5 g solid Lapatinib-base form X and 0.5 ml HCl 32%, 45 ml (30V) methanol was added to obtain a yellow suspension. The resulting suspension was stirred for 20 hours at 25° C. The cake thus obtained was dried for 16 hours at 40° C. in a vacuum oven, and identified as Form C1 of Lapatinib di-hydrochloride. Heating 50 mg of the obtained material yielded Form C1 of Lapatinib di-hydrochloride with higher crystallinity

### Example 7

To the mixture of 1.5 g solid Lapatinib-base form X and 0.584 ml HBr 48%, 45 ml (30V) methanol was added to obtain a yellow suspension. The resulting suspension was stirred for 20 hours at 25° C. The cake thus obtained was dried for 16 hours at 40° C. in a vacuum oven and identified as amorphous Lapatinib di-hydrobromide.

### Example 8

To the mixture of 0.5 gr solid Lapatinib-base Form X and 0.0615 ml phosphoric acid 85%, 25 ml (50V) methanol was added to obtain a yellow suspension. The resulting suspension was stirred for 9 hours at 25° C., then filtered. The cake thus obtained was dried for 16 hours at 40° C. in a vacuum oven, and identified as Form P2 of Lapatinib phosphate.

### Example 9

To the mixture of 0.5 gr solid Lapatinib-base Form X and 0.123 ml phosphoric acid 85%, 25 ml (50V) methanol was added to obtain a yellow suspension. The resulting suspension was stirred for 9 hours at 25° C., then filtered. The cake thus obtained was identified as Form P3 of Lapatinib phosphate.

### Example 10

The cake obtained by example 9 was dried for 16 hours at 40° C. in a vacuum oven, identified as Form P4 of Lapatinib phosphate.

### Example 11

To the mixture of 1.5 g solid Lapatinib base and 0.3 g Maleic acid, 45 ml (30V) of methanol was added to obtain a yellow suspension. The resulting suspension was stirred for 20 hours at 25° C., than filtered. The cake thus obtained was dried for 16 hours at 40° C. in a vacuum oven, identified as a mixture of Lapatinib maleate form L1 and un-reacted Lapatinib base form X. The mixture was heated to 150° C. for 30 minutes in an open glass vial. The sample was cooled to 25° C. and identified as form L1 of Lapatinib maleate.

### Example 12

To the mixture of 2 g solid Lapatinib base form X and 0.516 g L-tartaric acid, 60 ml (30V) of methanol was added to obtain a yellow suspension. The resulting suspension was stirred for 20 hours at 25° C., than filtered. The cake thus obtained was identified as a mixture of amorphous Lapatinib tartrate and un-reacted Lapatinib base form X. The mixture was heated to 70° C. for 30 minutes in an open glass vial. The sample was cooled to 25° C. and identified as amorphous Lapatinib tartrate.

### Example 13

Form U1 of lapatinib sulfate was heated to 150° C. for 30 minutes in an open glass vial. The sample was cooled to 25° C. and identified as amorphous lapatinib sulfate.

### Example 14

To the mixture of 9.02 gr solid Lapatinib-base, 0.82 ml (2 eq) Sulfuric acid 98%, 300 ml MeOH was added to obtain yellow suspension. The resulting suspension was stirred for 20 hours at 25° C., whereupon it was filtered. The cake thus obtained was dried for 48 hours, at 40° C. in a vacuum oven, identified as U3 form of Lapatinib sulfate.

### Example 15

0.5 gr solid Lapatinib sulfate Form U3 was mixed with 20V methyl-ethyl-ketone. The resulting suspension was stirred over 3 hours at 25° C., whereupon it was filtered. The wet sample was identified as form U4 of Lapatinib sulfate, as presented in FIG. 4.4. The cake thus obtained was dried for 16 hours, at 50° C. in a vacuum oven, identified as form U4 of Lapatinib sulfate, as presented in FIG. 4.5.

### Example 16

0.5 gr solid Lapatinib sulfate Form U3 was mixed with 20V acetone. The resulting suspension was stirred for 3 hours at 25° C., whereupon it was filtered. The wet sample was identified as form U5 of Lapatinib-sulfate, as presented in FIG. 4.6. The cake thus obtained was dried for 16 hours, at 50° C. in a vacuum oven, identified as form U5 of Lapatinib sulfate, as presented in FIG. 4.7.

### Example 17

0.5 gr solid Lapatinib sulfate Form U3 was mixed with 20V DMF. The resulting suspension was stirred for 3 hours at 25° C., whereupon it was filtered. The wet sample was identified as form U6 of Lapatinib sulfate, as presented in FIG. 4.8. The cake thus obtained was dried for 16 hours, at 50° C. in a vacuum oven, identified as U6 form of Lapatinib sulfate, as presented in FIG. 4.9.

### Example 18

0.5 gr solid Lapatinib sulfate Form U3 was mixed with 20V DMA. The resulting suspension was stirred over 3 hours at 25° C., whereupon it was filtered. The wet sample was identified as form U7 of Lapatinib sulfate, as presented in FIG. 4.11. The cake thus obtained was dried for 16 hours, at 50° C. in a vacuum oven, identified as U6 form of Lapatinib sulfate, as presented in FIG. 4.10.

### Example 19

0.5 gr solid Lapatinib sulfate Form U3 was mixed with 20V water. The resulting suspension was stirred for 3 hours at 25° C., whereupon it was filtered. The cake thus obtained was identified as amorphous Lapatinib sulfate as presented in FIG. 14.13.

### Example 20

To the 0.5 gr solid Lapatinib dihydrochloride Form C1, 10 ml H₂O were added to obtain a yellow suspension. The resulting suspension was stirred for 3 hours at 25° C., whereupon it was filtered. The obtained cake was dried for 16 hours at 60° C. in a vacuum oven, to obtain form C1 of lapatinib dihydrochloride.

### Example 21

To the 0.5 gr solid Lapatinib-HCl Form C1, 10 ml DMSO were added to obtain a yellow suspension. The resulting suspension was stirred for 3 hours at 25° C., whereupon it was filtered. The cake thus obtained was identified as Amorphous Lapatinib-HCl. The obtained cake was dried for 16 hours at 60° C. in a vacuum oven, to obtain form C1 of lapatinib-HCl.

What is claimed is:

1. Lapatinib monotosylate.

2. The Lapatinib monotosylate of claim 1, wherein the compound is solid.

3. The Lapatinib monotosylate of claim 1, wherein the compound is crystalline.

4. Lapatinib monotosylate Form M1, characterized by data selected from the group consisting of: a PXRD pattern with peaks at about 5.3, 6.0, 7.4, 10.0, and 17.7±0.2 degrees 2-theta; a PXRD pattern with peaks at about 5.3, 6.0, and 7.4±0.2 degrees 2-theta, and at least two peaks selected from the group consisting of 17.7, 18.6, 19.9, 23.3 and 23.9±0.2 degrees 2-theta; a PXRD pattern with peaks at about 5.2, 6.0, 7.4, 10.0, 17.6, 19.8, 21.5, 23.3, 23.9 and 26.7±0.2 degrees 2-theta; a solid-state ¹³C NMR spectrum with signals at about 113.3, 127.0 and 129.4±0.2 ppm; and a solid-state ¹³C NMR spectrum having chemical shifts in the range of 100 to 180 ppm of about 3.0, 16.7 and 19.1±0.1 ppm relative to a lowest chemical shift in the range of 110 to 180 ppm, wherein the lowest chemical shift is typically at about 110.3±0.1 ppm.

5. The Lapatinib monotosylate Form M1 of claim 4, characterized by a PXRD pattern with peaks at about 5.3, 6.0, 7.4, 10.0, and 17.7±0.2 degrees 2-theta.

6. The Lapatinib monotosylate Form M1 of claim 4, characterized by a PXRD pattern illustrated in FIG. 1.1.

7. A process for preparing the Lapatinib monotosylate Form M1 of claim 4 comprising forming a suspension of Lapatinib base with about 1 to about 1.2 equivalents of p-toluenesulfonic acid in methanol; and recovering the precipitate from the obtained slurry.

8. The process of claim 7, wherein the suspension is maintained at about 0° C. to about room temperature for about 16 hours to about 24 hours.

9. The process of claim 7, wherein the obtained precipitate is dried.

10. Lapatinib sulfate.

11. The Lapatinib sulfate of claim 10, wherein the compound is solid.

12. The Lapatinib sulfate of claim 10, wherein the compound is crystalline.

13. Lapatinib sulfate Form U6, characterized by a PXRD pattern with peaks at about 5.2, 10.4, 11.4 and 12.7±0.2 degrees 2-theta.

14. The Lapatinib sulfate Form U6 of claim 13, further characterized by a PXRD pattern with peaks at about 5.2, 7.5, 10.4, 11.4, 12.7, 18.1, 18.6, 19.5, 20.1 and 22.5±0.2 degrees 2-theta.

15. The Lapatinib sulfate Form U6 of claim 13, characterized by a PXRD pattern illustrated in FIG. 4.9.

16. A process for preparing the Lapatinib sulfate Form U6 of claim 13 comprising forming a suspension of lapatinib sulfate in a solvent selected from the group consisting of dimethylformamide (DMF), and dimethylacetamide (DMA); and recovering the precipitate from the obtained slurry, wherein, if DMA is used, the obtained precipitate is further dried.

17. The process of claim 16 wherein the suspension is maintained at about 0° C. to about room temperature for about 3 hours to about 24 hours.

18. The process of claim 16, wherein the obtained precipitate is further dried.

19. A process for preparing Lapatinib sulfate, comprising forming a reaction mixture by combining Lapatinib base and sulfuric acid; and recovering the obtained Lapatinib sulfate.

20. The process of claim 19, wherein the reaction mixture further comprises a solvent selected from the group consisting of C₁₋₈ alcohols, C₁₋₆ ketones, DMF, and DMA.

21. The process of claim 20, wherein the solvent is selected from the group consisting of methanol, acetone, dimethylformamide, and dimethylacetamide.

22. An amorphous Lapatinib sulfate.

23. The amorphous Lapatinib sulfate of claim 22, characterized by a PXRD pattern illustrated in FIG. 4.12.

24. A process for preparing the amorphous Lapatinib sulfate of claim 22 comprising heating Lapatinib sulfate to form a melt; and cooling the melt.

25. The process of claim 24, wherein the Lapatinib sulfate is heated to a temperature of about 150° C.

26. A process for preparing the amorphous Lapatinib sulfate of claim 22 comprising combining Lapatinib sulfate with water to form a suspension; and maintaining the suspension for about 3 hours to about 24 hours to obtain amorphous Lapatinib sulfate.

27. The process of claim 26, wherein the suspension is maintained at about 0° C. to about 35° C.

28. Lapatinib di-hydrochloride Form C1, characterized by data selected by the group consisting of: a PXRD pattern with peaks at about 7.7, 9.9, 11.8, 13.6 and 16.5±0.2 degrees 2-theta; a PXRD pattern with peaks at about 7.7, 9.9, 11.8, 13.6, 16.5, 17.9, 18.6, 20.5, 22.4 and 23.7±0.2 degrees 2-theta; a PXRD pattern with peaks at about 7.6, 10.0, and 11.8±0.2 degrees 2-theta, and at least two peaks selected from the group consisting of 16.5, 18.1, 20.4, 22.3 and 23.6±0.2 degrees 2-theta; a solid-state ¹³C NMR spectrum with signals at about 113.0, 131.0 and 152.3±0.2 ppm; and a solid-state ¹³C NMR spectrum having chemical shifts in the range of range of 100 to 180 ppm of about 3.5, 21.5 and 42.8±0.1 ppm relative to a lowest chemical shift in the range of 110 to 180 ppm, wherein the lowest chemical shift is typically at about 109.5±0.1 ppm.

29. The Lapatinib di-hydrochloride Form C1 of claim 28, characterized by a PXRD pattern illustrated in FIG. 5.1.

30. A process for preparing the lapatinib di-hydrochloride Form C1 of claim 28 comprising providing a suspension of Lapatinib di-hydrochloride in methanol; and recovering the precipitate.

31. The process of claim 30, wherein the Lapatinib di-hydrochloride is formed in situ, by combining Lapatinib and hydrochloric acid.

32. The process of claim 30, wherein the slurry is maintained at about 0° C. to about room temperature for about 3 hours to about 24 hours.

33. The process of claim 30, wherein the obtained precipitate is further dried.

34. Amorphous Lapatinib di-hydrobromide.

35. The amorphous Lapatinib di-hydrobromide of claim 34, characterized by a PXRD pattern illustrated in FIG. 6.

36. A process for preparing the amorphous Lapatinib di-hydrobromide of claim 34 comprising forming a suspension of lapatinib base with hydrobromic acid in methanol; and recovering the precipitate from the obtained slurry.

37. The process of claim 36 wherein the suspension is maintained at about 0° C. to about room temperature for about 16 hours to about 24 hours.

38. The process of claim 36, wherein the obtained precipitate is further dried.

39. Lapatinib phosphate.

40. The Lapatinib phosphate of claim 39, wherein the compound is solid.

41. The Lapatinib phosphate of claim 39, wherein the compound is crystalline.

42. A process for preparing amorphous Lapatinib tartrate comprising forming a suspension of lapatinib base with tartaric acid in methanol; and recovering the precipitate from the obtained slurry.

43. The process of claim 42, wherein the suspension is maintained at about 0° C. to about room temperature for about 16 hours to about 24 hours.

44. The process of claim 42, wherein the obtained precipitate is further dried.

45. A process for preparing the crystalline Form L1 of Lapatinib maleate comprising forming a suspension of lapatinib base with maleic acid in methanol; and recovering the precipitate from the obtained suspension.

46. The process of claim 45, wherein the suspension is maintained at about room temperature to about reflux for about 16 hours to about 24 hours.

47. The process of claim 45, wherein the obtained precipitate is further dried.

48. A process for preparing crystalline Form S1 of Lapatinib succinate comprising forming a suspension of lapatinib base with maleic acid in methanol; and recovering the precipitate from the obtained slurry.

49. The process of claim 48, wherein the suspension is maintained at about 0° C. to about room temperature for about 16 hours to about 24 hours.

50. The process of claim 48, wherein the obtained precipitate is further dried.

51. A pharmaceutical composition comprising the Lapatinib compound of any of claims 1, 4, 10, 13, 22, 28, 34, and 39, and at least one pharmaceutically acceptable excipient.

52. The pharmaceutical composition of claim 51, wherein the lapatinib compound is Lapatinib monotosylate or Lapatinib monotosylate Form M1

53. A pharmaceutical composition comprising the Lapatinib compounds made by the process of any of claims 7, 16, 19, 24, 26, 30, 36, 42, 45, and 48, and at least one pharmaceutically acceptable excipient.

54. A process for preparing a pharmaceutical formulation comprising combining the Lapatinib compound of any of claims 1, 4, 10, 13, 22, 28, 34, and 39, with at least one pharmaceutically acceptable excipient.

55. A process for preparing a pharmaceutical formulation comprising combining the Lapatinib compound made by the process of any of claims 7, 16, 19, 24, 26, 30, 36, 42, 45, and 48, with at least one pharmaceutically acceptable excipient.

56. Method of treating metastatic breast cancer comprising administrating to a patient a therapeutically effective amount of Lapatinib compound of any of claims 1, 4, 10, 13, 22, 28, 34, and 39.

57. Method of treating metastatic breast cancer comprising administrating to a patient a therapeutically effective amount of Lapatinib compound made by the process of any of claims 7, 16, 19, 24, 26, 30, 36, 42, 45, and 48.
